(19) United States
(12) Patent Application Publication
(10) Pub. No.: US 2019/0248765 A1
(43) Pub. Date: Aug. 15, 2019

(54) COMPOUNDS, COMPOSITIONS, AND METHODS FOR INCREASING CFTR ACTIVITY

(71) Applicant: Proteostasis Therapeutics, Inc., Boston, MA (US)

(72) Inventors: Daniel Parks, Pepperell, MA (US); Benito Munoz, Newtonville, MA (US)

(21) Appl. No.: 16/345,084

(22) PCT Filed: Oct. 26, 2017
(86) PCT No.: PCT/US2017/058469

§ 371 Date: Apr. 25, 2019

Related U.S. Application Data
(60) Provisional application No. 62/413,202, filed on Oct. 26, 2016.

(51) Int. Cl. C07D 403/06; C07D 417/14; C07D 409/14; C07D 471/04; A61P 11/00; C07D 413/06

(52) U.S. Cl. CPC C07D 403/06; C07D 417/14; C07D 413/06; C07D 471/04; A61P 11/00; C07D 409/14

(57) ABSTRACT

The present disclosure is directed to disclosed compounds that modulate, e.g., address underlying defects in cellular processing of CFTR activity.

### CROSS REFERENCE TO RELATED APPLICATIONS

This application is a national stage filing under 35 U.S.C. § 371 of PCT/US2017/058469, filed Oct. 26, 2017, which claims the benefit of, and priority to, U.S. provisional application Ser. No. 62/413,202, filed Oct. 26, 2016, the contents of which are hereby incorporated by reference herein in their entirety.

### BACKGROUND

Cells normally maintain a balance between protein synthesis, folding, trafficking, aggregation, and degradation, referred to as protein homeostasis, utilizing sensors and networks of pathways (Sitia et al., Nature 426: 891-894, 2003; Ron et al., Nat Rev Mol Cell Biol 8: 519-529, 2007). The cellular maintenance of protein homeostasis, or proteostasis, refers to controlling the conformation, binding interactions, location and concentration of individual proteins making up the proteome. Protein folding in vivo is accomplished through interactions between the folding polypeptide chain and macromolecular cellular components, including multiple classes of chaperones and folding enzymes, which minimize aggregation (Wiseman et al., Cell 131: 809-821, 2007). Whether a given protein folds in a certain cell type depends on the distribution, concentration, and subcellular localization of chaperones, folding enzymes, metabolites and the like (Wiseman et al.). Cystic fibrosis and other maladies of protein misfolding arise as a result of an imbalance in the capacity of the protein homeostasis (proteostasis) environment to handle the reduced energetic stability of misfolded, mutated proteins that are critical for normal physiology (Balch et al., Science 319, 916-9 (2008); Powers, et al., Annu Rev Biochem 78, 959-91 (2009); Hutt et al., FEBS Lett 583, 2639-46 (2009)).

Cystic Fibrosis (CF) is caused by mutations in the cystic fibrosis transmembrane conductance regulator (CFTR) gene which encodes a multi-membrane spanning epithelial chloride channel (Riordan et al., Annu Rev Biochem 77, 701-26 (2008)). Approximately ninety percent of patients have a deletion of phenylalanine (Phe) 508 (ΔF508) on at least one allele. This mutation results in disruption of the energetics of the protein fold leading to degradation of CFTR in the endoplasmic reticulum (ER). The ΔF508 mutation is thus associated with defective folding and trafficking, as well as enhanced degradation of the mutant CFTR protein (Qu et al., J Biol Chem 272, 15739-44 (1997)). The loss of a functional CFTR channel at the plasma membrane disrupts ionic homeostasis (Cl⁻, Na⁺, HCO₃⁻) and airway surface hydration leading to reduced lung function (Riordan et al.). Reduced periciliary liquid volume and increased mucus viscosity impede mucociliary clearance resulting in chronic infection and inflammation, phenotypic hallmarks of CF disease (Boucher, J Intern Med 261, 5-16 (2007)). In addition to respiratory dysfunction, ΔF508 CFTR also impacts the normal function of additional organs (pancreas, intestine, gall bladder), suggesting that the loss-of-function impacts multiple downstream pathways that will require correction.

In addition to cystic fibrosis, mutations in the CFTR gene and/or the activity of the CFTR channel has also been implicated in other conditions, including for example, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), dry eye disease, Sjogren's syndrome and chronic sinusitis, cholestatic liver disease (e.g. Primary biliary cirrhosis (PBC) and primary sclerosing cholangitis (PSC)) (Sloane et al. (2012), PLoS ONE 7(6): e39809.doi:10.1371/journal. pone.0039809; Bombieri et al. (2011), J Cyst Fibros. 2011 June; 10 Suppl 2:S86-102; (Albert et al. (2008), Clinical Respiratory Medicine, Third Ed., Mosby Inc.; Levin et al. (2005), Invest Ophthalmol Vis Sci., 46(4):1428-34; Froussard (2007), Pancreas 35(1): 94-5), Son et al. (2017) J Med Chem 60(6):2401-10.

There remains a need in the art for compounds, compositions and methods of increasing CFTR activity as well as for methods of treating CF, other CFTR-related diseases, and other maladies of protein misfolding.

### SUMMARY

This disclosure is directed in part to compounds represented by:

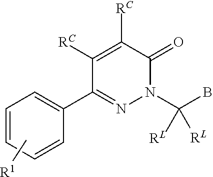

and pharmaceutically acceptable salts thereof, in which B, R¹, R^{C}, and R^{L} are as defined herein.

Also contemplated herein are pharmaceutical compositions that include a disclosed compound such as those compounds having disclosed formulas and a pharmaceutically acceptable carrier or excipient. In certain embodiments, the compositions can include at least one additional CFTR modulator, for example, may include one, two, three, four, five or more additional CFTR modulators.

In certain embodiments, a method is provided comprising administering a disclosed compound to a subject (e.g., a human patient) suffering from a disease associated with decreased CFTR activity (e.g., cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, A-β-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, Sjogren's syndrome, familial hypercholesterolemia, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, nephrogenic DI, Charcot-Marie Tooth syndrome, Perlizaeus-Merzbacher disease, Alzheimer's disease, Parkinson's disease, amyotrophic lateral sclerosis, progressive supranuclear palsy, Pick's disease, Huntington's disease, spinocerebellar ataxia type I, spinal and bulbar muscular atrophy, dentatorubral pallidoluysian, myotonic dystrophy, hereditary Creutzfeldt-Jakob disease (due to prion protein processing defect), Fabry disease, cholestatic liver disease (e.g. Primary biliary cirrhosis (PBC) and primary sclerosing cholangitis (PSC)), and Straussler-Scheinker syndrome. In certain embodiments, the disease is cystic fibrosis. For example, contemplated herein is a method for treating a patient suffering from cystic fibrosis comprising administering to said patient an effective amount of a disclosed compound.

In some embodiments, disclosed methods described herein can further include administering at least one additional CFTR modulator e.g., administering at least two, three, four or five additional CFTR modulators. In certain embodiments, at least one additional CFTR modulator is a CFTR corrector (e.g., VX-809, VX-661, VX-659 and VX-983) or potentiator (e.g., ivacaftor and genistein). In certain of these embodiments, one of the at least two additional therapeutic agents is a CFTR corrector (e.g., VX-809, VX-661, VX-659 and VX-983) and the other is a CFTR potentiator (e.g., ivacaftor and genistein).

### DETAILED DESCRIPTION

As used herein, the words “a” and “an” are meant to include one or more unless otherwise specified. For example, the term “an agent” encompasses both a single agent and a combination of two or more agents.

As discussed above, the present disclosure is directed in part to compounds as described herein or a pharmaceutically acceptable salt, prodrug or solvate thereof, pharmaceutical compositions, methods of increasing CFTR activity and methods of treating cystic fibrosis.

For example, provided herein are compounds represented by:

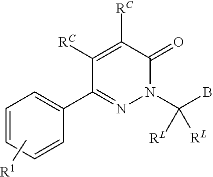

or pharmaceutically acceptable salts and/or stereoisomers thereof, wherein:

Ring B is selected from the group consisting of a 5-6 membered monocyclic heteroaryl having 1, 2 or 3 heteroatoms each independently selected from the group consisting of S, N, NR^{a} and O; a 9-10 membered bicyclic heteroaryl having 1, 2 or 3 heteroatoms each selected from the group consisting of S, N, NR^{a} and O;

R¹ independently for each occurrence, is selected from the group consisting of hydrogen, halogen, hydroxyl, cyano, C₁₋₆alkyl, C₂₋₆alkenyl, C₂₋₆alkynyl, C₃₋₆cycloalkyl, heterocycle, C₁₋₆alkoxy, —NR^{a}R^{b}, phenyl, benzyl, and —O-phenyl;

Ring B is optionally substituted by one, two, three or four substituents each selected from R⁶;

R⁶ is, independently for each occurrence, selected from the group consisting of halogen, hydroxyl, cyano, C₁₋₆alkyl, C₂₋₆alkenyl, C₂₋₆alkynyl, C₃₋₆cycloalkyl, heterocycle, R⁶⁶, C₁₋₆alkoxy, and —NR^{a}R^{b};

R⁶⁶ is phenyl or a 5 membered heteroaryl with 1, 2 or 3 heteroatoms each selected from O, S, and N, wherein R⁶⁶ is optionally substituted with one or two substituents each selected from halogen, hydroxyl, cyano, C₁₋₆alkyl, C₂₋₆alkenyl, C₂₋₆alkynyl, C₁₋₆alkoxy, C₃₋₆cycloalkyl, and heterocycle;

R^{C} is independently selected for each occurrence from the group consisting of H, halogen, hydroxyl, C₁₋₆alkyl, C₁₋₆alkoxy, C₃₋₆cycloalkyl, phenyl and —O-phenyl;

R^{L} is independently selected for each occurrence from the group consisting of H, methyl, ethyl, propyl, butyl, cyclopropyl, cyclobutyl, cyclohexyl, cyclopentyl, heteroaryl, heterocycle, phenyl and benzyl;

R^{a} is independently selected for each occurrence from the group consisting of H, C₁₋₃alkyl, C₃₋₆ cycloalkyl, phenyl, and C(O)—C₁₋₃alkyl;

R^{b} is independently selected for each occurrence from the group consisting of H and C₁₋₃alkyl; or R^{a} and R^{b} taken together with the nitrogen to which they are attached form a 3-6 membered heterocyclic ring; and

wherein C₁₋₆alkyl, C₂₋₆alkenyl, C₂₋₆alkynyl, C₃₋₆cycloalkyl, C₁₋₆alkoxy, C₃₋₆cycloalkyl, heteroaryl, heterocycle, benzyl and phenyl are each optionally substituted by one, two or three substituents each independently selected from halogen, cyano, methyl, methoxy, carboxy, C(O)—O—C₁₋₃ alkyl, C(O)—C₁₋₃alkyl phenyl, —NR^{a}R^{b}, S(O)_{w}-methyl (where w is 0, 1 or 2), —S(O)_{w}—NR^{a}R^{b}(where w is 0, 1 or 2), and —NR^{b}—S(O)_{w} (where w is 0, 1, or 2), and hydroxyl.

In certain embodiments, ring B may be:

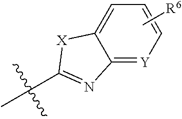

wherein X is O or NR^{a} and Y is CH or N.

In certain embodiments, ring B may have at least one N member and one NR^{a} member.

For example, ring B may be:

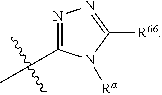

In certain embodiments, R⁶⁶ may be phenyl or 5 membered heteroaryl.

In an embodiment, one R^{L} may be H and one R^{L} may be methyl. In a further embodiment, R^{C} for each occurrence may be selected from H and halogen.

In certain embodiments, B may be selected from the group consisting of:

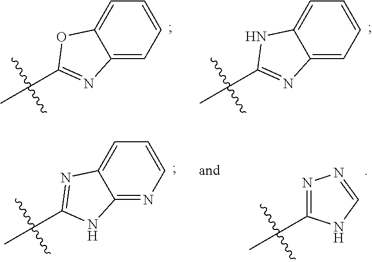 [SMILES: *c1nc2ccccc2o1 ; *c1nc2ccccc2[nH]1 ; *c1nc2cccnc2[nH]1 ; *c1nnc[nH]1]

For example, a disclosed compound may be selected from the group consisting of:

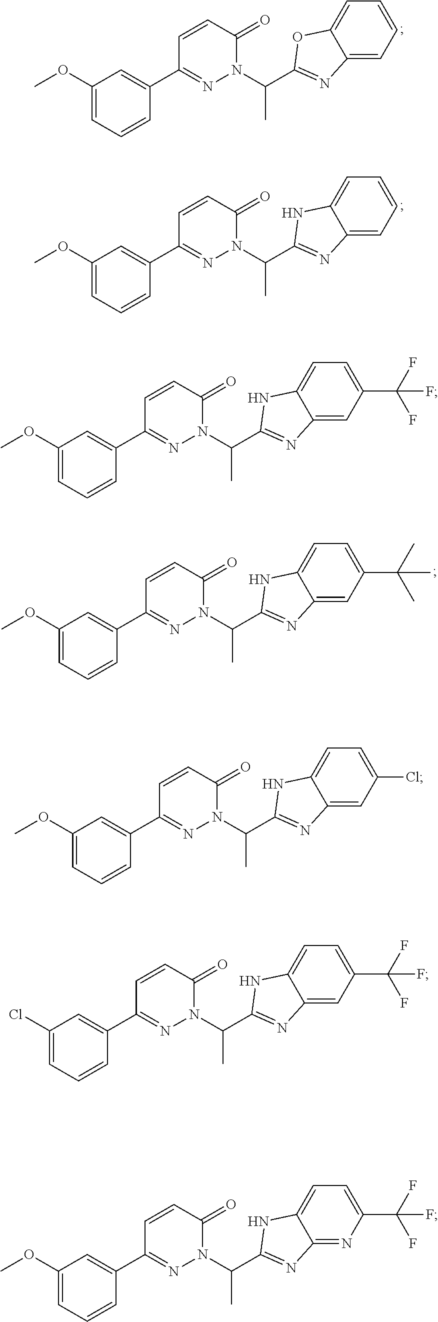

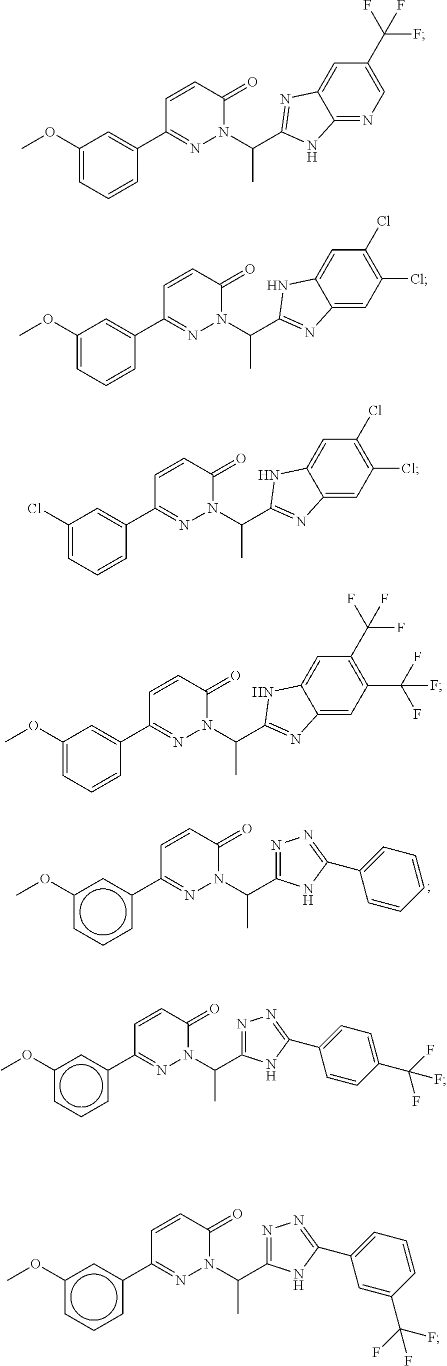

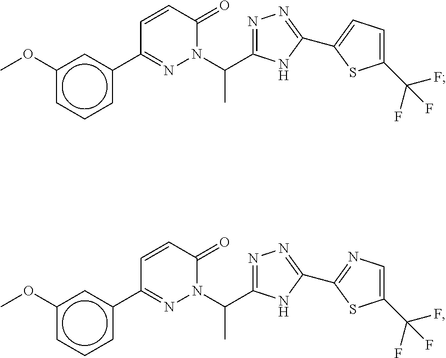

and a pharmaceutically acceptable salt or stereoisomer thereof.

Also provided herein are compounds disclosed in the Exemplification.

Also contemplated herein are pharmaceutical compositions that include a disclosed compound and a pharmaceutically acceptable carrier or excipient. In certain embodiments, the compositions can include at least one additional CFTR modulator as described anywhere herein or at least two additional CFTR modulators, each independently as described anywhere herein.

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

It will be appreciated that the description of the present disclosure herein should be construed in congruity with the laws and principals of chemical bonding.

The term “alkyl”, as used herein, unless otherwise indicated, refers to both branched and straight-chain saturated aliphatic hydrocarbon groups having the specified number of carbon atoms; for example, “C₁-C₁₀ alkyl” denotes alkyl having 1 to 10 carbon atoms, and straight or branched hydrocarbons of 1-6, 1-4, or 1-3 carbon atoms, referred to herein as C₁₋₆ alkyl, C₁₋₄ alkyl, and C₁₋₃ alkyl, respectively. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

The term “carbonyl” as used herein refers to the radical —C(O)—.

The term “cyano” as used herein refers to the radical —CN.

The term, “alkenyl”, as used herein, refers to both straight and branched-chain moieties having the specified number of carbon atoms and having at least one carbon-carbon double bond. Exemplary alkenyl groups include, but are not limited to, a straight or branched group of 2-6 or 3-4 carbon atoms, referred to herein as C₂₋₆ alkenyl, and C₃₋₄ alkenyl, respectively. Exemplary alkenyl groups include, but are not limited to, vinyl, allyl, butenyl, pentenyl, etc.

The term, “alkynyl”, as used herein, refers to both straight and branched-chain moieties having the specified number or carbon atoms and having at least one carbon-carbon triple bond.

The term “cycloalkyl,” as used herein, refers to saturated cyclic alkyl moieties having 3 or more carbon atoms, for example, 3-10, 3-6, or 4-6 carbons, referred to herein as C₃₋₁₀ cycloalkyl, C₃₋₆ cycloalkyl or C₄₋₆ cycloalkyl, respectively for example. Unless otherwise stated, such saturated cyclic alkyl moieties can contain up to 18 carbon atoms and include monocycloalkyl, polycycloalkyl, and benzocycloalkyl structures. Monocycloalkyl refers to groups having a single ring group. Polycycloalkyl denotes hydrocarbon systems containing two or more ring systems with one or more ring carbon atoms in common; i.e., a spiro, fused, or bridged structure. Benzocycloalkyl signifies a monocyclic alkyl group fused to a benzene ring, referred to herein as C₈₋₁₂benzocycloalkyl, for example. Examples of monocycloalkyl groups include, but are not limited to, cyclopropyl, cyclobutyl, cyclopentyl, cyclohexyl, cycloheptyl, cyclooctyl, cyclononyl, cyclodecyl, cycloundecyl, cyclododecyl, cyclotridecyl, cyclotetradecyl, cyclopentadecyl, cyclohexadecyl, cycloheptadecyl, and cyclooctadecyl. Examples of polycycloalkyl groups include, but are not limited to, decahydronaphthalene, spiro[4.5]decyl, bicyclo[2.2.1]heptyl, bicyclo[3.2.1]octyl, pinanyl, norbornyl, adamantyl, and bicyclo[2.2.2]octyl. Examples of benzocycloalkyl groups include, but are not limited to, tetrahydronaphthyl, indanyl, and 1.2-benzocycloheptanyl.

The term “cycloalkoxy” refers to a cycloalkyl group as just described, that is a monocycloalkyl, polycycloalkyl, or benzocycloalkyl structure, bound to the remainder of the molecule through an ethereal oxygen atom. Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, etc. The term “benzocycloalkoxy” refers to a monocyclic cycloalkoxy group fused to a benzene ring, referred to herein for example as C₈₋₁₂benzocycloalkoxy. Examples of benzocycloalkoxy groups include, but are not limited to, tetrahydronaphthyloxy, indanyloxy, and 1.2-benzocycloheptanyloxy.

The term “cycloalkenyl,” as used herein, refers to cyclic alkenyl moieties having 3 or more carbon atoms.

The term “cycloalkynyl,” as used herein, refers to cyclic alkynyl moieties having 5 or more carbon atoms.

“Alkylene” means a straight or branched, saturated aliphatic divalent radical having the number of carbons indicated. “Cycloalkylene” refers to a divalent radical of carbocyclic saturated hydrocarbon group having the number of carbons indicated.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆ alkoxy, and C₂₋₆ alkoxy, respectively. Exemplary alkoxy groups include, but are not limited to methoxy, ethoxy, isopropoxy, etc.

The term “alkoxyalkyl” as used herein refers to a straight or branched alkyl group attached to oxygen, attached to a second straight or branched alkyl group (alkyl-O-alkyl-). Exemplary alkoxyalkyl groups include, but are not limited to, alkoxyalkyl groups in which each of the alkyl groups independently contains 1-6 carbon atoms, referred to herein as C₁₋₆alkoxy-C₁₋₆alkyl. Exemplary alkoxyalkyl groups include, but are not limited to methoxymethyl, 2-methoxyethyl, 1-methoxyethyl, 2-methoxypropyl, ethoxymethyl, 2-isopropoxyethyl etc.

The term “alkyoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆ alkoxycarbonyl. Exemplary alkoxycarbonyl groups include, but are not limited to, methoxycarbonyl, ethoxycarbonyl, t-butoxycarbonyl, etc.

The term “alkenyloxy” used herein refers to a straight or branched alkenyl group attached to oxygen (alkenyl-O—). Exemplary alkenyloxy groups include, but are not limited to, groups with an alkenyl group of 3-6 carbon atoms, referred to herein as C₃₋₆alkenyloxy. Exemplary “alkenyloxy” groups include, but are not limited to allyloxy, butenyloxy, etc.

The term “alkynyloxy” used herein refers to a straight or branched alkynyl group attached to oxygen (alkynyl-O). Exemplary alkynyloxy groups include, but are not limited to, groups with an alkynyl group of 3-6 carbon atoms, referred to herein as C₃₋₆alkynyloxy. Exemplary alkynyloxy groups include, but are not limited to, propynyloxy, butynyloxy, etc.

The term “heterocyclic” or “heterocycle” encompasses heterocycloalkyl, heterocycloalkenyl, heterobicycloalkyl, heterobicycloalkenyl, heteropolycycloalkyl, heteropolycycloalkenyl, and the like unless indicated otherwise. Heterocycloalkyl refers to cycloalkyl groups containing one or more heteroatoms (O, S, or N) within the ring. Heterocycloalkenyl as used herein refers to cycloalkenyl groups containing one or more heteroatoms (O, S or N) within the ring. Heterobicycloalkyl refers to bicycloalkyl groups containing one or more heteroatoms (O, S or N) within a ring. Heterobicycloalkenyl as used herein refers to bicycloalkenyl groups containing one or more heteroatoms (O, S or N) within a ring, a heterocycle can refer to, for example, a saturated or partially unsaturated 4- to 12 or 4-10-membered ring structure, including monocyclic, bridged bicyclic, fused bicyclic and spirocyclic rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran, etc.

The term “oxo” as used herein refers to the radical ═O.

Cycloalkyl, cycloalkenyl, and heterocyclic groups also include groups similar to those described above for each of these respective categories, but which are substituted with one or more oxo moieties.

The term “heteroaryl”, as used herein, refers to aromatic carbocyclic groups containing one or more heteroatoms (O, S, or N) within a ring. A heteroaryl group, unless indicated otherwise, can be monocyclic or polycyclic. A heteroaryl group may additionally be substituted or unsubstituted. Contemplated heteroaryl groups include ring systems substituted with one or more oxo moieties. A polycyclic heteroaryl can comprise fused rings, covalently attached rings or a combination thereof. A polycyclic heteroaryl is a polycyclic ring system that comprises at least one aromatic ring containing one or more heteroatoms within a ring. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, thiazolopyridinyl, oxazolopyridinyl and azaindolyl. The foregoing heteroaryl groups may be C-attached or heteroatom-attached (where such is possible). For instance, a group derived from pyrrole may be pyrrol-1-yl (N-attached) or pyrrol-3-yl (C-attached). In some embodiments, the heteroaryl is 4- to 12-membered heteroaryl. In yet other embodiments, the heteroaryl is a mono or bicyclic 4- to 10-membered heteroaryl.

The term “heterocyclyloxy” as used herein refers to a heterocyclyl group attached to oxygen (heterocyclyl-O—).

The term “heteroaryloxy” as used herein refers to a heteroaryl group attached to oxygen (heteroaryl-O—).

The terms “halo” or “halogen” as used herein refer to F, Cl, Br, or I.

The term “haloalkyl” as used herein refers to an alkyl group having 1 to (2n+1) substituent(s) independently selected from F, Cl, Br or I, where n is the maximum number of carbon atoms in the alkyl group. It will be understood that haloalkyl is a specific example of an optionally substituted alkyl.

The terms “hydroxy” and “hydroxyl” as used herein refers to the radical —OH.

As will be understood by the skilled artisan, “H” is the symbol for hydrogen, “N” is the symbol for nitrogen, “S” is the symbol for sulfur, “0” is the symbol for oxygen. “Me” is an abbreviation for methyl.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of disclosed compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diasterisomers of disclosed compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009. Where a particular compound is described or depicted, it is intended to encompass that chemical structure as well as tautomers of that structure.

The term “enantiomerically pure” means a stereomerically pure composition of a compound. For example, a stereochemically pure composition is a composition that is free or substantially free of other stereoisomers of that compound. In another example, for a compound having one chiral center, an enantiomerically pure composition of the compound is free or substantially free of the other enantiomer. In yet another example, for a compound having two chiral centers, an enantiomerically pure composition is free or substantially free of the other diastereomers.

Where a particular stereochemistry is described or depicted it is intended to mean that a particular enantiomer is present in excess relative to the other enantiomer. A compound has an R-configuration at a specific position when it is present in excess compared to the compound having an S-configuration at that position. A compound has an S-configuration at a specific position when it is present in excess compared to the compound having an R-configuration at that position.

The compounds disclosed herein can exist in solvated as well as unsolvated forms with pharmaceutically acceptable solvents such as water, ethanol, and the like, and it is intended that disclosed compounds include both solvated and unsolvated forms. In one embodiment, a disclosed compound is amorphous or, in another embodiment, a single polymorph. In another embodiment, a disclosed compound is a mixture of polymorphs. In another embodiment, a disclosed compound is in a crystalline form.

Isotopically labeled compounds are also contemplated herein, which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a disclosed compound may have one or more H atoms replaced with deuterium.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly suitable for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be suitable in some circumstances. Isotopically labeled compounds can generally be prepared by following procedures analogous to those disclosed in the examples herein by substituting an isotopically labeled reagent for a non-isotopically labeled reagent.

In some embodiments one or more of the nitrogen atoms of a disclosed compound if present are oxidized to N-oxide.

Representative synthetic routes for the preparation of the compounds disclosed herein are provided throughout the Examples section. As will be understood by the skilled artisan, diastereomers can be separated from the reaction mixture using column chromatography.

Disclosed compounds may be also be prepared using methods described in the literature, including, but not limited to, J. Med. Chem. 2011, 54(13), 4350-64; Russian Journal of Organic Chemistry, 2011, 47(8), 1199-1203; U.S. Patent Application Publication No. 2009/0036451 A1; WO2008/046072 A2, and U.S. Pat. No. 4,336,264, the contents of each of which are expressly incorporated by reference herein.

As discussed above, contemplated herein in an embodiment is a method of increasing CFTR activity in a subject comprising administering an effective amount of a disclosed compound. Also contemplated herein is a method of treating a patient suffering from a condition associated with CFTR activity comprising administering to said patient an effective amount of a compound described herein.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “subject” is an animal to be treated or in need of treatment. A “patient” is a human subject in need of treatment.

An “effective amount” refers to that amount of an agent that is sufficient to achieve a desired and/or recited effect. In the context of a method of treatment, an “effective amount” of the therapeutic agent that is sufficient to ameliorate of one or more symptoms of a disorder and/or prevent advancement of a disorder, cause regression of the disorder and/or to achieve a desired effect.

The term “modulating” encompasses increasing, enhancing, inhibiting, decreasing, suppressing, and the like. The terms “increasing” and “enhancing” mean to cause a net gain by either direct or indirect means. As used herein, the terms “inhibiting” and “decreasing” encompass causing a net decrease by either direct or indirect means.

In some examples, CFTR activity is enhanced after administration of a compound described herein when there is an increase in the CFTR activity as compared to that in the absence of the administration of the compound. CFTR activity encompasses, for example, chloride channel activity of the CFTR, and/or other ion transport activity (for example, HCO₃⁻ transport). In certain of these embodiments, the activity of one or more (e.g., one or two) mutant CFTRs (e.g., ΔF508, S549N, G542X, G551D, R117H, N1303K, W1282X, R553X, 621+1G>T, 1717-1G>A, 3849+10kbC>T, 2789+5G>A, 3120+1G>A, I507del, R1162X, 1898+1G>A, 3659delC, G85E, D1152H, R560T, R347P, 2184insA, A455E, R334W, Q493X, and 2184delA CFTR) is enhanced (e.g., increased). Contemplated patients may have a CFTR mutation(s) from one or more classes, such as without limitation, Class I CFTR mutations, Class II CFTR mutations, Class III CFTR mutations, Class IV CFTR mutations, Class V CFTR mutations, and Class VI mutations. Contemplated subject (e.g., human subject) CFTR genotypes include, without limitation, homozygote mutations (e.g., ΔF508/ΔF508 and R117H/R117H) and compound heterozygote mutations (e.g., ΔF508/G551D; ΔF508/A455E; ΔF508/G542X; A508F/W1204X; R553X/W1316X; W1282X/N1303K, 591A18/E831X, F508del/R117H/N1303K/3849+10kbC>T; A303K/384; and DF508/G178R).

In certain embodiments, the mutation is a Class I mutation, e.g., a G542X; a Class II/I mutation, e.g., a ΔF508/G542X compound heterozygous mutation. In other embodiments, the mutation is a Class III mutation, e.g., a G551D; a Class II/Class III mutation, e.g., a ΔF508/G551D compound heterozygous mutation. In still other embodiments, the mutation is a Class V mutation, e.g., a A455E; Class II/Class V mutation, e.g., a ΔF508/A455E compound heterozygous mutation. Of the more than 1000 known mutations of the CFTR gene, ΔF508 is the most prevalent mutation of CFTR which results in misfolding of the protein and impaired trafficking from the endoplasmic reticulum to the apical membrane (Dormer et al. (2001). J Cell Sci 114, 4073-4081; http://www.genet.sickkids.on.ca/app). In certain embodiments, ΔF508 CFTR activity is enhanced (e.g., increased). In certain embodiments, ΔF508 CFTR activity and/or G542X CFTR activity and/or G551D CFTR activity and/or A455E CFTR activity is enhanced (e.g., increased). An enhancement of CFTR activity can be measured, for example, using literature described methods, including for example, Ussing chamber assays, patch clamp assays, and hBE Ieq assay (Devor et al. (2000), Am J Physiol Cell Physiol 279(2): C461-79; Dousmanis et al. (2002), J Gen Physiol 119(6): 545-59; Bruscia et al. (2005), PNAS 103(8): 2965-2971).

As discussed above, the disclosure also encompasses a method of treating cystic fibrosis. Methods of treating other conditions associated with CFTR activity, including conditions associated with deficient CFTR activity, comprising administering an effective amount of a disclosed compound, are also provided herein.

For example, provided herein is a method of treating a condition associated with deficient or decreased CFTR activity comprising administering an effective amount of a disclosed compound that enhances CFTR activity. Non-limiting examples of conditions associated with deficient CFTR activity are cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, Aβ-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

In some embodiments, disclosed methods of treatment further comprise administering an additional therapeutic agent. For example, in an embodiment, provided herein is a method of administering a disclosed compound and at least one additional therapeutic agent. In certain aspects, a disclosed method of treatment comprises administering a disclosed compound, and at least two additional therapeutic agents. Additional therapeutic agents include, for example, mucolytic agents, bronchodilators, antibiotics, anti-infective agents, anti-inflammatory agents, ion channel modulating agents, therapeutic agents used in gene therapy, CFTR correctors, and CFTR potentiators, or other agents that modulates CFTR activity. In some embodiments, at least one additional therapeutic agent is selected from the group consisting of a CFTR corrector and a CFTR potentiator. Non-limiting examples of CFTR correctors and potentiators include VX-770 (Ivacaftor), deuterated Ivacaftor, GLPG2851, GLPG2737, GLPG2451, VX-809 (3-(6-(1-(2,2-difluorobenzo[d][1,3]dioxol-5-yl)cyclopropanecarboxamido)-3-methylpyridin-2-yl)benzoic acid, VX-661 (1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide), VX-983, VX-152, VX-440, VX-659, and Ataluren (PTC124) (3-[5-(2-fluorophenyl)-1,2,4-oxadiazol-3-yl]benzoic acid), FDL169, GLPG1837/ABBV-974 (for example, a CFTR potentiator), GLPG2665, GLPG2222 (for example, a CFTR corrector); N-(4-(tert-butyl)-2-(tert-butyldimethylsilyl)-5-hydroxyphenyl)-4-oxo-1,4-dihydroquinoline-3-carboxamide N-(4-(tert-butyl)-2-(tert-butyldimethylsilyl)-5-hydroxyphenyl)-4-oxo-1,4-dihydroquinoline-3-carboxamide, and compounds described in, e.g., WO2014/144860 and 2014/176553, hereby incorporated by reference. Non-limiting examples of modulators include QBW-251, QR-010, NB-124, riociquat, and compounds described in, e.g., WO2014/045283; WO2014/081821, WO2014/081820, WO2014/152213; WO2014/160440, WO2014/160478, US2014027933; WO2014/0228376, WO2013/038390, WO2011/113894, WO2013/038386; and WO2014/180562, of which the disclosed modulators in those publications are contemplated as an additional therapeutic agent and incorporated by reference. Non-limiting examples of anti-inflammatory agents include N6022 (3-(5-(4-(1H-imidazol-1-yl) phenyl)-1-(4-carbamoyl-2-methylphenyl)-1H-pyrrol-2-yl) propanoic acid), CTX-4430, N1861, N1785, and N91115.

In some embodiments, the methods described herein can further include administering an additional therapeutic agent or administering at least two additional CFTR therapeutic agents. In some embodiments, the methods described herein can further include administering an additional CFTR modulator or administering at least two additional CFTR modulators. In certain embodiments, at least one CFTR modulator is a CFTR corrector (e.g., VX-809, VX-661, VX-983, VX-152, VX-440, VX-659, and GLPG2222 or GLPG2665) or potentiator (e.g., ivacaftor, genistein and GLPG1837). In certain of these embodiments, one of the at least two additional therapeutic agents is a CFTR corrector (e.g., VX-809, VX-661, VX-152, VX-440, VX-659 and VX-983) and the other is a CFTR potentiator (e.g., ivacaftor and genistein). In certain of these embodiments, one of the at least two additional therapeutic agents is a CFTR corrector (e.g., GLPG2222) and the other is a CFTR potentiator (e.g., GLPG1837). In certain of these embodiments, one of the at least two additional therapeutic agents is a CFTR corrector (e.g., VX-809 or VX-661) and the other is a CFTR potentiator (e.g., ivacaftor). In certain of these embodiments, at least one CFTR modulator is an agent that enhances read-through of stop codons (e.g., NB124 or ataluren). NB124 has the structure:

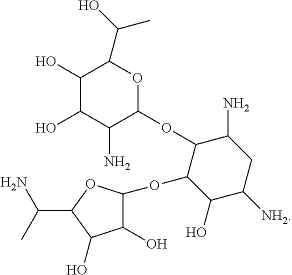 [SMILES: CC(N)C1OC(OC2C(O)C(N)CC(N)C2OC2OC(C(C)O)C(O)C(O)C2N)C(O)C1O]

In other embodiments, the methods described herein can further include administrating an epithelial sodium channel (ENaC) inhibitor (e.g., VX-371).

Accordingly, in another aspect, this disclosure provides a method of treating a condition associated with deficient or decreased CFTR activity (e.g., cystic fibrosis), which includes administering to a subject in need thereof (e.g., a human patient in need thereof) an effective amount of a disclosed compound and at least one or two additional CFTR therapeutic agent(s) (e.g., at least one or two additional CFTR therapeutic agents, e.g., in which one of the at least one or two additional therapeutic agents is optionally a CFTR corrector, modulator or amplifier (e.g., VX-809, VX-661, VX-983, VX-659, VX-152, VX-440, GLPG2222, NB124, ataluren, sodium 5-((1R)-1-(tetrahydro-2H-pyran-4-yl)ethoxy)-8-methyl-2-(3-methyl-1-benzofuran-2-yl)quinoline-4-carboxylate) and/or the other is a CFTR potentiator (e.g., ivacaftor, genistein, N-(4-(tert-butyl)-2-(tert-butyldimethylsilyl)-5-hydroxyphenyl)-4-oxo-1,4-dihydroquinoline-3-carboxamide, and GLPG1837); e.g., one of the at least two additional therapeutic agents is GLPG2222, and the other is GLPG1837; or one of the at least two additional therapeutic agents is VX-809 or VX-661, and the other is ivacaftor. Additional agents, e.g. amplifiers, are disclosed in co-pending applications PCT/US14/044100, PCT/US15/020460, PCT/US15/020499, and PCT/US15/036691, each incorporated by reference. For example, an exemplary amplifier is N-(3-(5-(hydroxymethyl)-1H-1,2,3-triazol-1-yl)propyl)-5-phenylisoxazole-3-carboxamide (“Compound A”). In certain embodiments, the subject's CFTR genotype includes, without limitation, one or more Class I CFTR mutations, one or more Class II CFTR mutations, one or more Class III CFTR mutations, one or more Class IV CFTR mutations, or one or more Class V CFTR mutations, or one or more Class VI CFTR mutations. In certain embodiments, the subject's CFTR genotype includes, without limitation, one or more homozygote mutations (e.g., ΔF508/ΔF508 or R117H/R117H) and/or one or more compound heterozygote mutations (e.g., ΔF508/G551D; ΔF508/A455E; ΔF508/G542X; A508F/W1204X; R553X/W1316X; W1282X/N1303K; F508del/R117H; N1303K/3849+10kbC>T; ΔF508/R334W; DF508/G178R, and 591A18/E831X). In certain embodiments, the subject's CFTR genotype includes a Class I mutation, e.g., a G542X Class I mutation, e.g., a ΔF508/G542X compound heterozygous mutation. In other embodiments, the subject's CFTR genotype includes a Class III mutation, e.g., a G551D Class III mutation, e.g., a ΔF508/G551D compound heterozygous mutation. In still other embodiments, the subject's CFTR genotype includes a Class V mutation, e.g., a A455E Class V mutation, e.g., a ΔF508/A455E compound heterozygous mutation. In certain embodiments, ΔF508 CFTR activity and/or G542X CFTR activity and/or G551D CFTR activity and/or A455E activity is enhanced (e.g., increased). In certain embodiments, the enhancement in activity (e.g., increase in activity) provided by the combination of the disclosed compound and one or two additional therapeutic agents is greater than additive when compared to the enhancement in activity provided by each therapeutic component individually.

| Class | Effect on CFTR protein | Example of mutation |
|---|---|---|
| I | Shortened protein | W1282X Instead of inserting the |
| | | amino acid tryptophan (W), the |
| | | protein sequence is prematurely |
| | | stopped (indicated by an X). |
| II | Protein fails to reach cell | ΔF508 A phenylalanine amino |
| | membrane | acid (F) is deleted |
| III | Channel cannot be regulated | G551D A “missense” mutation: |
| | properly | instead of a glycine amino acid |
| | | (G), aspartate (D) is added |
| IV | Reduced chloride conductance | R117H Missense |
| V | Reduced due to incorrect | 3120 + 1G > A Splice-site |
| | splicing of gene | mutation in gene intron 16 |
| VI | Reduced due to protein | N287Y a A -> T at 991 |
| | instability | |

| Genotype | Description | Possible Symptoms |
|---|---|---|
| Δ508F/Δ508F | homozygote | Severe lung disease, |
| | | pancreatic insufficient |
| R117H/R117H | homozygote | Congenital bilateral |
| | | absence of the vas |
| | | deferens, |
| | | No lung or pancreas |
| | | disease, |
| WT/Δ508F | heterozygote | Unaffected |
| WT/3120 + 1 G > A | heterozygote | Unaffected |
| Δ508F/W1204X | compound heterozygote | No lung disease, |
| | | pancreatic insufficient |
| R553X and W1316X | compound heterozygote | Mild lung disease, |
| | | pancreatic insufficient |
| 591Δ18/E831X | compound heterozygote | No lung or pancreas |
| | | disease, nasal polyps |

For example, provided herein is a method of treating a patient having one or more of the following mutations in the CFTR gene: G1244E, G1349D, G178R, G551S, S1251N, S1255P, S549N, S549R, G970R, or R117H, and/or e.g., a patient with one or two copies of the F508del mutation, or one copy of the ΔF508 mutation and a second mutation that results in a gating effect in the CFTR protein (e.g., a patient that is heterozygous for ΔF508 and G551D mutation), a patient with one copy of the ΔF508 mutation and a second mutation that results in residual CFTR activity, or a patient with one copy of the ΔF508 mutation and a second mutation that results in residual CFTR activity, comprising administering an effective amount of a disclosed compound. As described herein, such exemplary methods (e.g., of a patient having one or mutations such as those described above) may include, for example, administering to such patient a combination therapy, e.g., administering (simultaneously or sequentially) an effective amount of ivacaftor to said patient and an effective amount of a disclosed compound that may act as an amplifier or a disclosed compound that may act as a corrector. Such administration may result, for example, in increased chloride transport in human bronchial epithelial cells with e.g., one or two copies of mutations, e.g, ΔF508 mutation, as compared to administration of ivacaftor alone. Another combination therapy that includes a disclosed compound may also include an effective amount of a readthrough agent (e.g., ataluren, NB124) and an effective amount of disclosed compound that may act as an amplifier or as a corrector.

Without being limited by theory, a disclosed compound may be advantageous as compared to known CFTR modulators. For example, a disclosed compound may exhibit cAMP-dependent activity in F508del-CFTR HBE cells with continuous exposure, as evidenced, e.g., in an Ussing Chamber Assay with F508del/F508del HBE cells treated with an amplifier for 24 hr in the presence of a disclosed compound or acutely with ivacaftor. For example, a disclosed compound may exhibit acute potentiator activity additive to lumacaftor in F508del-CFTR HBE cells, as evidenced in an Ussing Chamber Assay with F508del/F508del HBE cells treated acutely with a disclosed compound or ivacaftor after 24 hr lumacaftor treatment. A disclosed compound's activity may be, e.g., additive with lumacaftor with continuous exposure in patient intestinal organoids. For example, a disclosed compound's peak efficacy on F508del-CFTR may be comparable to ivacaftor under chronic conditions and exhibits superior time-dependent activity, as evidenced in an Ussing Chamber Assay with F508del/F508del HBE cells treated with lumacaftor for 24 hr in the presence of a disclosed compound or ivacaftor or with acute ivacaftor. For example, a disclosed compound may exhibit low efficacy on G551D-CFTR, as evidenced, e.g., in an Ussing Chamber Assay with G551D/F508del HBE cells. For example, a disclosed compound's efficacy on conductance mutants R347P- and R117H-CFTR may comparable to, e.g., ivacaftor.

The phrase “combination therapy,” as used herein, refers to an embodiment where a patient is co-administered a disclosed compound, a CFTR potentiator agent (e.g., ivacaftor) and optionally, one or more CFTR corrector agent(s) (e.g, VX-661 and/or lumacaftor) as part of a specific treatment regimen intended to provide the beneficial effect from the co-action of these therapeutic agents. For example, a beneficial effect of a combination may include, but is not limited to, pharmacokinetic or pharmacodynamic co-action resulting from the combination of therapeutic agents. For example, administration of a disclosed compound with ivacaftor alone or with a CFTR corrector agent (e.g., lumacaftor or VX-661) may result in a level of function (e.g., as measured by chloride activity in HBE cells or patients that have a ΔF508 mutation, that achieves clinical improvement (or better) as compared to the chloride activity level in cells or patients with a G551D mutation receiving ivacaftor alone, or ivacaftor and a corrector agent (lumacaftor or VX-661; or for example, administration of a disclosed compound with ivacaftor alone or ivacaftor with a CFTR corrector agent (e.g., lumacaftor or VX-661) may result in a level of function (e.g., as measured by chloride activity in HBE cells or patients that have a A455E mutation, that achieves clinical improvement (or better) as compared to the chloride activity level at e.g., 50% or more of wild type cells; or upon administration of a disclosed compound and ivacaftor to a patient (e.g. having a G551D class III mutation) may show e.g., about two times or more improved activity of ivacaftor as compared to administration of ivacaftor alone. Administration of disclosed therapeutic agents in combination typically is carried out over a defined time period (usually a day, days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, inhalational routes, intravenous routes, intramuscular routes, and direct absorption through mucous membrane tissues. The therapeutic agents can be administered by the same route or by different routes. For example, a first therapeutic agent of the combination selected may be administered by intravenous injection or inhalation or nebulizer while the other therapeutic agents of the combination may be administered orally. Alternatively, for example, all therapeutic agents may be administered orally or all therapeutic agents may be administered by intravenous injection, inhalation or nebulization.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by a day, days or even weeks.

The components of a disclosed combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

In a further aspect, a method of identifying a candidate agent that increases CFTR activity is provided, which includes: (i) contacting a cell that expresses a CFTR protein with the candidate agent and a disclosed compound; (ii) measuring the CFTR activity in the cell in the presence of the candidate agent and the disclosed compound; and (iii) comparing the CFTR activity to that in the absence of the test agent, wherein an increase in CFTR activity in the presence of the test agent indicates that the agent increases CFTR activity. In certain embodiments, the cell expresses a mutant CFTR protein. In certain embodiments, CFTR activity is measured by measuring chloride channel activity of the CFTR, and/or other ion transport activity. In certain of these embodiments, the method is high-throughput. In certain of these embodiments, the candidate agent is a CFTR corrector or a CFTR potentiator.

The term “pharmaceutically acceptable salt(s)” as used herein refers to salts of acidic or basic groups that may be present in a disclosed compounds used in disclosed compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, e.g., calcium, magnesium, sodium, lithium, zinc, potassium, and iron salts. Examples of such salts also include, e.g., ammonium salts and quaternary ammonium salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

In an embodiment, contemplated methods may include for example, administering prodrugs of the compounds described herein, for example, prodrugs of a disclosed compound, or a pharmaceutical composition thereof.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255). For example, if a compound of the disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino-(C₂₋₃)alkyl (such as β-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a compound of the disclosure contains an alcohol functional group, a prodrug can be formed by the replacement of the hydrogen atom of the alcohol group with a group such as (C₁₋₆) alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxy)methyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplicio, et al., Molecules 2008, 13, 519 and references therein.

Also contemplated in certain embodiments is the use of clathrates of the compounds described herein, pharmaceutical compositions comprising the clathrates, and methods of use of the clathrates. Clathrates of a disclosed compound or a pharmaceutical composition thereof are also contemplated herein.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

As discussed above, the disclosure also contemplates administration of pharmaceutical compositions comprising a pharmaceutically acceptable carrier or excipient and a compound described herein. A disclosed compound, or a pharmaceutically acceptable salt, solvate, clathrate or prodrug thereof, can be administered in pharmaceutical compositions comprising a pharmaceutically acceptable carrier or excipient. The excipient can be chosen based on the expected route of administration of the composition in therapeutic applications. The route of administration of the composition depends on the condition to be treated. For example, intravenous injection may be suitable for treatment of a systemic disorder and oral administration may be suitable to treat a gastrointestinal disorder. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. A pharmaceutical composition comprising a disclosed compound or a pharmaceutically acceptable salt, solvate, clathrate or prodrug, can be administered by a variety of routes including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

Disclosed compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are suitable liquid carriers, particularly for injectable solutions.

Injectable formulations can be prepared either as liquid solutions or suspensions; solid forms suitable for solution in, or suspension in, liquid vehicles prior to injection can also be prepared. The preparation also can also be emulsified or encapsulated in liposomes or micro particles such as polylactide, polyglycolide, or copolymer for enhanced adjuvant effect, as discussed above [Langer, Science 249: 1527, 1990 and Hanes, Advanced Drug Delivery Reviews 28: 97-119, 1997]. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, transdermal applications and ocular delivery. For suppositories, binders and carriers include, for example, polyalkylene glycols or triglycerides; such suppositories can be formed from mixtures containing the active ingredient in the range of about 0.5% to about 10%, or about 1% to about 2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

For the purpose of oral therapeutic administration, the pharmaceutical compositions can be incorporated with excipients and used in the form of tablets, troches, capsules, elixirs, suspensions, syrups, wafers, chewing gums and the like. Tablets, pills, capsules, troches and the like may also contain binders, excipients, disintegrating agent, lubricants, glidants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used. In another embodiment, the composition is administered as a tablet or a capsule.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like. For vaginal administration, a pharmaceutical composition may be presented as pessaries, tampons, creams, gels, pastes, foams or spray.

The pharmaceutical composition can also be administered by nasal administration. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compounds prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administered to the skin.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present disclosure, “pulmonary” will also mean to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

The disclosure also encompasses the treatment of a condition associated with a dysfunction in proteostasis in a subject comprising administering to said subject an effective amount of a disclosed compound that enhances, improves or restores proteostasis of a protein. Proteostasis refers to protein homeostasis. Dysfunction in protein homeostasis is a result of protein misfolding, protein aggregation, defective protein trafficking or protein degradation. For example, the disclosure contemplates s administering a disclosed compound that corrects protein misfolding, reduces protein aggregation, corrects or restores protein trafficking and/or affects protein degradation for the treatment of a condition associated with a dysfunction in proteostasis. In some aspects, a disclosed compound that corrects protein misfolding and/or corrects or restores protein trafficking is administered. In cystic fibrosis, the mutated or defective enzyme is the cystic fibrosis transmembrane conductance regulator (CFTR). One of the most common mutations of this protein is ΔF508 which is a deletion (A) of three nucleotides resulting in a loss of the amino acid phenylalanine (F) at the 508th (508) position on the protein. As described above, mutated cystic fibrosis transmembrane conductance regulator exists in a misfolded state and is characterized by altered trafficking as compared to the wild type CFTR. Additional exemplary proteins of which there can be a dysfunction in proteostasis, for example that can exist in a misfolded state, include, but are not limited to, glucocerebrosidase, hexosamine A, aspartylglucosaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α-N-acetylgalactosaminidase, α-neuramidase, β-glucuronidase, β-hexosamine A and acid lipase, polyglutamine, α-synuclein, TDP-43, superoxide dismutase (SOD), Δβ peptide, tau protein, transthyretin and insulin. The compounds of the disclosure can be used to restore proteostasis (e.g., correct folding and/or alter trafficking) of the proteins described above.

Protein conformational diseases encompass gain of function disorders and loss of function disorders. In one embodiment, the protein conformational disease is a gain of function disorder. The terms “gain of function disorder,” “gain of function disease,” “gain of toxic function disorder” and “gain of toxic function disease” are used interchangeably herein. A gain of function disorder is a disease characterized by increased aggregation-associated proteotoxicity. In these diseases, aggregation exceeds clearance inside and/or outside of the cell. Gain of function diseases include, but are not limited to, neurodegenerative diseases associated with aggregation of polyglutamine, Lewy body diseases, amyotrophic lateral sclerosis, transthyretin-associated aggregation diseases, Alzheimer's disease, Machado-Joseph disease, cerebral B-amyloid angiopathy, retinal ganglion cell degeneration, tauopathies (progressive supranuclear palsy, corticobasal degeneration, frontotemporal lobar degeneration), cerebral hemorrhage with amyloidosis, Alexander disease, Serpinopathies, familial amyloidotic neuropathy, senile systemic amyloidosis, ApoAI amyloidosis, ApoAII amyloidosis, ApoAIV amyloidosis, familial amyloidosis of the Finnish type, lysozyme amyloidosis, fibrinogen amyloidosis, dialysis amyloidosis, inclusion body myositis/myopathy, cataracts, medullary thyroid carcinoma, cardiac atrial amyloidosis, pituitary prolactinoma, hereditary lattice corneal dystrophy, cutaneous lichen amyloidosis, corneal lactoferrin amyloidosis, corneal lactoferrin amyloidosis, pulmonary alveolar proteinosis, odontogenic tumor amyloid, seminal vesical amyloid, sickle cell disease, critical illness myopathy, von Hippel-Lindau disease, spinocerebellar ataxia 1, Angelman syndrome, giant axon neuropathy, inclusion body myopathy with Paget disease of bone, frontotemporal dementia (IBMPFD) and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Δβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease, lewy body dementia (LBD) and multiple system atrophy (SMA). Prion diseases (also known as transmissible spongiform encephalopathies or TSEs) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia and Kuru. In another embodiment, the misfolded protein is alpha-1 anti-trypsin.

In a further embodiment, the protein conformation disease is a loss of function disorder. The terms “loss of function disease” and “loss of function disorder” are used interchangeably herein. Loss of function diseases are a group of diseases characterized by inefficient folding of a protein resulting in excessive degradation of the protein. Loss of function diseases include, for example, lysosomal storage diseases. Lysosomal storage diseases are a group of diseases characterized by a specific lysosomal enzyme deficiency which may occur in a variety of tissues, resulting in the build-up of molecules normally degraded by the deficient enzyme. The lysosomal enzyme deficiency can be in a lysosomal hydrolase or a protein involved in the lysosomal trafficking. Lysosomal storage diseases include, but are not limited to, aspartylglucosaminuria, Fabry's disease, Batten disease, Cystinosis, Farber, Fucosidosis, Galactasidosialidosis, Gaucher's disease (including Types 1, 2 and 3), Gml gangliosidosis, Hunter's disease, Hurler-Scheie's disease, Krabbe's disease, α-Mannosidosis, f3-Mannosidosis, Maroteaux-Lamy's disease, Metachromatic Leukodystrophy, Morquio A syndrome, Morquio B syndrome, Mucolipidosis II, Mucolipidosis III, Neimann-Pick Disease (including Types A, B and C), Pompe's disease, Sandhoff disease, Sanfilippo syndrome (including Types A, B, C and D), Schindler disease, Schindler-Kanzaki disease, Sialidosis, Sly syndrome, Tay-Sach's disease and Wolman disease.

In another embodiment, a disease associated with a dysfunction in proteostasis is a cardiovascular disease. Cardiovascular diseases include, but are not limited to, coronary artery disease, myocardial infarction, stroke, restenosis and arteriosclerosis. Conditions associated with a dysfunction of proteostasis also include ischemic conditions, such as, ischemia/reperfusion injury, myocardial ischemia, stable angina, unstable angina, stroke, ischemic heart disease and cerebral ischemia.

In yet another embodiment, a treatment of a disease associated with a dysfunction in proteostasis is diabetes and/or complications of diabetes, including, but not limited to, diabetic retinopathy, cardiomyopathy, neuropathy, nephropathy, and impaired wound healing is contemplated.

In a further embodiment, a treatment of a disease associated with a dysfunction in proteostasis is an ocular disease including, but not limited to, age-related macular degeneration (AMD), diabetic macular edema (DME), diabetic retinopathy, glaucoma, cataracts, retinitis pigmentosa (RP) and dry macular degeneration is contemplated.

In yet additional embodiments, a disclosed method is directed to treating a disease associated with a dysfunction in proteostasis, wherein the disease affects the respiratory system or the pancreas. In certain additional embodiments, a contemplated method encompass treating a condition selected from the group consisting of polyendocrinopathy/hyperinsulinemia, diabetes mellitus, Charcot-Marie Tooth syndrome, Pelizaeus-Merzbacher disease, and Gorham's Syndrome.

Additional conditions associated with a dysfunction of proteostasis include hemoglobinopathies, inflammatory diseases, intermediate filament diseases, drug-induced lung damage and hearing loss. For example, provided herein are methods for the treatment of hemoglobinopathies (such as sickle cell anemia), an inflammatory disease (such as inflammatory bowel disease, colitis, ankylosing spondylitis), intermediate filament diseases (such as non-alcoholic and alcoholic fatty liver disease) and drug induced lung damage (such as methotrexate-induced lung damage). In another embodiment, methods for treating hearing loss, such as noise-induced hearing loss, aminoglycoside-induced hearing loss, and cisplatin-induced hearing loss comprising administering a disclosed compound are provided.

Additional conditions include those associated with a defect in protein trafficking and that can be treated according to a disclosed methods include: PGP mutations, hERG trafficking mutations, nephrongenic diabetes insipidus mutations in the arginine-vasopressin receptor 2, persistent hyperinsulinemic hypoglycemia of infancy (PHH1) mutations in the sulfonylurea receptor 1, and α1AT.

The disclosure is illustrated by the following examples which are not meant to be limiting in any way.

### EXEMPLIFICATION

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. At least some of the compounds identified as “intermediates” herein are contemplated as compounds of the disclosure.

List of Abbreviations

| Abbreviation | Name |
|---|---|
| rt | room temperature |
| THF | tetrahydrofuran |
| MeCN | acetonitrile |
| DMSO | dimethylsulfoxide |
| DCM | dichloromethane |
| EtOH | ethanol |
| MeOH | methanol |
| IPA | isopropanol |
| tBuOH | tert-butanol |
| EtOAc | ethyl acetate |
| DMF | N,N-dimethylformamide |
| TFA | trifluoroacetic acid |
| AcOH | acetic acid |
| HATU | 1-[Bis(dimethylamino)methylene]-1H-1,2,3-triazolo[4,5- |
| | b]pyridinium-3-oxide hexafluorophosphate |
| DIEA | N,N-diisopropylethylamine |
| TEA | triethylamine |
| dppf | 1,1′-bis(diphenylphosphino)ferrocene |
| atm | atmosphere |

### Example 1: 2-[1-(1,3-benzoxazol-2-yl)ethyl]-6-(3-methoxyphenyl)-2,3-dihydropyridazin-3-one (Compound 1)

A. 6-(3-Methoxyphenyl)-2,3-dihydropyridazin-3-one. To a 1000-mL round-bottom flask purged and maintained with an inert atmosphere of nitrogen was placed a solution of 6-chloro-2,3-dihydropyridazin-3-one (5 g, 38.30 mmol) and (3-methoxyphenyl)boronic acid (7.6 g, 50.01 mmol) in dioxane (300 mL)/water (15 mL) then Pd(dppf)Cl₂ (1.41 g) and K₂CO₃ (15.9 g, 115.04 mmol) were added. The reaction was stirred at 110° C. for 15 h, quenched by the addition of 100 mL of water, and extracted with EtOAc (3×150 mL). The organic extracts were combined, washed with brine (3×200 mL), dried over anhydrous Na₂SO₄, and concentrated under reduced pressure. The residue was purified by column chromatography eluting with EtOAc/petroleum ether (9:1) affording 5.6 g (72%) of the title compound as a white solid. Mass Spectrum (LCMS, ESI pos): Calcd. for C₁₁H₁₁N₂O₂+: 203.1 (M+H); Found: 203.1.

B. Ethyl 2-[3-(3-Methoxyphenyl)-6-oxo-1,6-dihydropyridazin-1-yl]propanoate. To a 250-mL round-bottom flask was placed a solution of 6-(3-methoxyphenyl)-2,3-dihydropyridazin-3-one (2 g, 9.89 mmol, as prepared in the previous step) and ethyl 2-chloropropanoate (4.08 g, 29.87 mmol) in acetone (100 mL) then K₂CO₃ (4.14 g, 29.95 mmol) was added. The reaction was stirred at 70° C. for 3 d then the solids were filtered out and the filtrate was concentrated under reduced pressure. The residue was purified by column chromatography eluting with EtOAc/petroleum ether (9:1) affording 2.5 g (84%) of the title compound as a white solid. Mass Spectrum (LCMS, ESI pos): Calcd. for C₁₆H₁₉N₂O₄⁺: 303.1 (M+H); Found: 303.1.

C. 2-[3-(3-Methoxyphenyl)-6-oxo-1,6-dihydropyridazin-1-yl]propanoic acid. To a 500-mL round-bottom flask, was placed a solution of ethyl 2-[3-(3-methoxyphenyl)-6-oxo-1,6-dihydropyridazin-1-yl]propanoate (5 g, 16.54 mmol, as prepared in the previous step) in MeOH (150 mL) then NaOH (1.98 g, 49.50 mmol) in water (30 mL) was added. The reaction was stirred for 2 h at rt then concentrated under reduced pressure. The resulting solution was washed with EtOAc (2×30 mL) then the pH value of the aqueous layer was adjusted to 2 with conc. HCl. The solids were collected by filtration and dried affording 4.06 g (90%) of the title compound as a white solid. Mass Spectrum (LCMS, ESI pos): Calcd. for C₁₄H₁₅N₂O₄⁺: 275.1 (M+H); Found: 275.1. ¹H NMR (300 MHz, DMSO-d₆): δ 10.03 (s, 1H), 8.12 (d, J=8.0 Hz, 1H), 7.49-7.41 (m, 3H), 7.12-7.04 (m, 2H), 5.47-5.42 (m, 1H), 3.83 (s, 3H), 1.64-1.62 (d, J=7.6 Hz, 1H.

D. 2-Nitrophenyl 2-[3-(3-Methoxyphenyl)-6-oxo-1,6-dihydropyridazin-1-yl]propanoate. To a 25-mL round-bottom flask was placed a solution of 2-[3-(3-methoxyphenyl)-6-oxo-1,6-dihydropyridazin-1-yl]propanoic acid (200 mg, 0.73 mmol, as prepared in the previous step) in DMF (5 mL) then 2-nitrophenol (122 mg, 0.88 mmol), HATU (418 mg, 1.10 mmol), and DIEA (284 mg, 2.20 mmol) were added. The reaction was stirred for 16 h at rt, diluted with 50 mL of water, and extracted with EtOAc (3×20 mL). The organic extracts were combined, dried over anhydrous Na₂SO₄, and concentrated under reduced pressure. The residue was purified by column chromatography eluting with EtOAc/petroleum ether (1:10 up to 1:1) affording 215 mg (75%) of the title compound as a yellow solid. Mass Spectrum (LCMS, ESI pos): Calcd. for C₂₀H₁₈N₃O₆⁺: 396.1 (M+H); Found: 396.1.

E. 2-[1-(1,3-Benzoxazol-2-yl)ethyl]-6-(3-methoxyphenyl)-2,3-dihydropyridazin-3-one. To a 25-mL round-bottom flask was placed a solution of 2-nitrophenyl 2-[3-(3-methoxyphenyl)-6-oxo-1,6-dihydropyridazin-1-yl]propanoate (150 mg, 0.38 mmol, as prepared in the previous step) in AcOH (4 mL) then Fe powder (161 mg, 2.88 mmol) was added. The reaction was stirred at 90° C. for 16 h then the solids were filtered out and the filtrate was concentrated under reduced pressure. The crude product was purified by Prep-HPLC (Waters:Column:X Bridge C18 OBD Prep Column, 100 Å, 5 μm, 19 mm×250 mm; Mobile Phase A: Water (0.05% TFA), Mobile Phase B: ACN; Flow rate: 20 mL/min; Gradient: 35% B to 75% B in 6 min; 254/220 nm) affording 4.1 mg (3%) of the title compound as an off-white solid. Mass Spectrum (LCMS, ESI pos): Calcd. for C₂₀H₁₈N₃O₃⁺: 348.1 (M+H); Found: 348.1. ¹H NMR (400 MHz, DMSO-d₆): δ 8.12-8.10 (m, 1H), 7.77-7.68 (m, 2H), 7.38-7.33 (m, 4H), 7.19-7.13 (m, 2H), 6.97-6.96 (m, 1H), 6.47-6.42 (q, J=6.8 Hz, 1H), 3.63 (s, 3H), 1.90-1.89 (d, J=6.8 Hz, 3H). HPLC purity (254 nm): 98.6%.

### Example 2: 2-[1-(1H-1,3-benzodiazol-2-yl)ethyl]-6-(3-methoxyphenyl)-2,3-dihydropyridazin-3-one (Compound 2)

A. N-(2-aminophenyl)-2-[3-(3-methoxyphenyl)-6-oxo-1,6-dihydropyridazin-1-yl]propanamide. To a 25-mL round-bottom flask was placed a solution of 2-[3-(3-methoxyphenyl)-6-oxo-1,6-dihydropyridazin-1-yl]propanoic acid (200 mg, 0.73 mmol, as prepared in Example 1, Step C) in DMF (5 mL) then benzene-1,2-diamine (118 mg, 1.09 mmol), HATU (418 mg, 1.10 mmol), and DIEA (284 mg, 2.20 mmol) were added. The reaction was stirred for 16 h at rt, diluted with water (50 mL), and extracted with EtOAc (2×30 mL). The organic extracts were combined, dried over anhydrous Na₂SO₄, and concentrated under reduced pressure. The residue was purified by column chromatography eluting with EtOAc/petroleum ether (1:10 up to 1:1) affording 180 mg (68%) of the title compound as a yellow solid. Mass Spectrum (LCMS, ESI pos): Calcd. for C₂₀H₂₁N₄O₃⁺: 365.2 (M+H); Found: 365.2.

B. 2-[1-(1H-1,3-Benzodiazol-2-yl)ethyl]-6-(3-methoxyphenyl)-2,3-dihydropyridazin-3-one. To a 25-mL round-bottom flask was placed a solution of N-(2-aminophenyl)-2-[3-(3-methoxyphenyl)-6-oxo-1,6-dihydropyridazin-1-yl]propanamide (180 mg, 0.49 mmol, as prepared in the previous step) in AcOH (5 mL) then the resulting solution was stirred at 80° C. for 6 h. The reaction was concentrated under reduced pressure and the residue was purified by Prep-HPLC (Waters: Column: X bridge Phenyl OBD Column, 5 um, 19*150 mm; Mobile Phase A: Water (10 mmol/L NH₄HCO₃), Mobile Phase B: ACN; Flow rate: 20 mL/min; Gradient: 30% B to 48% B in 6 min; 254/220 nm) affording 80.0 mg (47%) of the title compound as a white solid. Mass Spectrum (LCMS, ESI pos): Calcd. for C₂₀H₁₉N₄O₂⁺: 347.2 (M+H); Found: 346.9. ¹H NMR (300 MHz, DMSO-d₆): δ 12.39 (s, 1H), 8.11-8.08 (d, J=9.6 Hz, 1H), 7.57-7.49 (m, 2H), 7.42-7.32 (m, 2H), 7.24 (s, 1H), 7.16-7.11 (m, 3H), 6.99-6.96 (m, 1H), 6.44-6.37 (q, J=6.9 Hz, 1H), 3.62 (s, 3H), 1.90-1.88 (d, J=6.9 Hz, 3H). HPLC purity (254 nm): 97.1%.

Using the procedure described in Example 2, with reagents, starting materials, and conditions familiar to those skilled in the art, the following compounds representative of the disclosure were prepared:

| Compound | Name and Data |
|---|---|
| 3 | 6-(3-Methoxyphenyl)-2-(1-(5-(trifluoromethyl)-1H-benzo[d]imidazol-2- |
| | yl)ethyl)pyridazin-3(2H)-one. Mass Spectrum (LCMS, ESI pos): Calcd. for |
| | C₂₁H₁₈F₃N₄O₂⁺: 415.1 (M + H); Found: 415.3. ¹H NMR (400 MHz, DMSO-d₆): δ |
| | 12.85 (s, 1H), 8.11-8.09 (d, J = 9.6 Hz, 1H), 7.96 (s, 1H), 7.79-7.76 (m, 1H), 7.64-7.62 (m, |
| | 1H), 7.50-7.44 (m, 1H), 7.37-7.31 (m, 2H), 7.19 (s, 1H), 7.14-7.11 (d, J = 9.6 Hz, 1H), |
| | 6.97-6.95 (m, 1H), 6.44-6.39 (m, 1H), 3.63 (s, 3H), 1.90-1.88 (d, J = 6.8 Hz, 3H). |
| | HPLC purity (254 nm): 98.6%. |
| 4 | 2-(1-(5-(tert-Butyl)-1H-benzo[d]imidazol-2-yl)ethyl)-6-(3-methoxyphenyl)pyridazin- |
| | 3(2H)-one. Mass Spectrum (LCMS, ESI pos): Calcd. for C₂₄H₂₇N₄O₂⁺: 403.2 (M + H); |
| | Found: 403.3. ¹H NMR (300 MHz, DMSO-d₆): δ 12.22 (s, 1H), 8.09-8.06 (d, J = 10.0 Hz, |
| | 1H), 7.39-7.31 (m, 4H), 7.22-7.21 (m, 2H), 7.11-7.09 (d, J = 10.0 Hz, 1H), |
| | 6.97-6.95 (m, 1H), 6.37-6.35 (m, 1H), 3.63 (s, 3H), 1.87-1.85 (d, J = 6.8 Hz 3H), 1.30 (s, 9H). |
| | HPLC purity (254 nm): 99.5%. |
| 5 | 2-(1-(5-Chloro-1H-benzo[d]imidazol-2-yl)ethyl)-6-(3-methoxyphenyl)pyridazin-3(2H)- |
| | one. Mass Spectrum (LCMS, ESI pos): Calcd. for C₂₀H₁₈ClN₄O₂⁺: 381.1 (M + H); |
| | Found: 381.2. ¹H NMR (400 MHz, DMSO-d₆): δ 8.09 (s, 1H), 7.62 (s, 1H), |
| | 7.57-7.55 (m, 1H), 7.38-7.32 (m, 2H), 7.25-7.23 (m, 2H), 7.13-7.11 (d, J = 9.6 Hz, 1H), |
| | 6.99-6.95 (m, 1H), 6.43-6.38 (q, J = 6.8 Hz, 1H), 3.67 (s, 3H), 1.89-1.87 (d, J = 7.2 Hz, 3H). |
| | HPLC purity (254 nm): 99.1%. |
| 6 | 6-(3-Chlorophenyl)-2-(1-(5-(trifluoromethyl)-1H-benzo[d]imidazol-2- |
| | yl)ethyl)pyridazin-3(2H)-one. Mass Spectrum (LCMS, ESI pos): Calcd. for |
| | C₂₀H₁₅ClF₃N₄O₂⁺: 419.1 (M + H); Found: 419.3. ¹H NMR (300 MHz, DMSO-d₆): δ |
| | 12.85 (s, 1H), 8.17-8.14 (d, J = 9.6 Hz, 1H), 7.88-7.67 (m, 4H), 7.49-7.46 (m, 3H), |
| | 7.17-7.14 (d, J = 9.6 Hz, 1H), 6.45-6.43 (m, 1H), 1.93-1.91 (d, J = 6.9 Hz, 3H). HPLC |
| | purity (254 nm): 95.8%. |
| 7 | 6-(3-Methoxyphenyl)-2-(1-(5-(trifluoromethyl)-1H-imidazo[4,5-b]pyridin-2- |
| | yl)ethyl)pyridazin-3(2H)-one. Mass Spectrum (LCMS, ESI pos): Calcd. for |
| | C₂₀H₁₇F₃N₅O₂⁺: 416.1 (M + H); Found: 416.1. ¹H NMR (300 MHz, DMSO-d₆): δ |
| | 8.12 (d, J = 9.6 Hz, 2H), 7.64 (d. J = 8.1 Hz, 1H), 7.38-7.35 (m, 2H), 7.24 (s, 1H), 7.13 (d, J = 9.6 Hz, |
| | 1H), 6.99-6.97 (m, 1H), 6.44-6.42 (m, 1H), 3.66 (s, 3H), 1.91 (d, J = 7.2 Hz, |
| | 3H). HPLC purity (254 nm): 96.6%. |
| 8 | 6-(3-Methoxyphenyl)-2-(1-(6-(trifluoromethyl)-3H-imidazo[4,5-b]pyridin-2- |
| | yl)ethyl)pyridazin-3(2H)-one. Mass Spectrum (LCMS, ESI pos): Calcd. for |
| | C₂₀H₁₇F₃N₅O₂⁺: 416.1 (M + H); Found: 416.3. ¹H NMR (300 MHz, DMSO-d₆): δ |
| | 13.49 (br s, 1H), 8.67 (s, 1H), 8.37 (s, 1H), 8.12 (d, J = 9.9 Hz, 1H), 7.39-7.32 (m, 2H), |
| | 7.22 (s, 1H), 7.15-7.12 (m, 1H), 7.00-6.96 (m, 1H), 6.43 (q, J = 6.9 Hz, 1H), 3.66 (s, 3H), |
| | 1.92 (d, J = 6.9 Hz, 3H). HPLC purity (254 nm): 99.4%. |
| 9 | 2-(1-(5,6-Dichloro-1H-benzo[d]imidazol-2-yl)ethyl)-6-(3-methoxyphenyl)pyridazin- |
| | 3(2H)-one. Mass Spectrum (LCMS, ESI pos): Calcd. for C₂₀H₁₇Cl₂N₄O₂⁺: |
| | 415.1 (M + H); Found: 415.1. ¹H NMR (400 MHz, CD₃OD): δ 8.08-8.06 (m, 1H), 7.85 (s, |
| | 2H), 7.38 (brs, 2H), 7.29 (brs, 1H), 7.16 (d, J = 8.8 Hz, 1H), 7.02 (s, 1H), 6.58 (brs, 1H), |
| | 3.88 (s, 3H), 2.08 (s, 3H). HPLC purity (254 nm): 99.5%. |
| 10 | 6-(3-Chlorophenyl)-2-(1-(5,6-dichloro-1H-benzo[d]imidazol-2-yl)ethyl)pyridazin- |
| | 3(2H)-one. Mass Spectrum (LCMS, ESI pos): Calcd. for C₁₉H₁₄Cl₂N₄O⁺: 419.0 (M + H); |
| | Found: 418.9. ¹H NMR (400 MHz, DMSO-d₆): δ 12.75 (s, 1H), 8.15 (d, J = 9.6 Hz, |
| | 1H), 7.88 (s, 1H), 7.83 (s, 1H), 7.79-7.77 (m, 1H), 7.69 (s, 1H), 7.50-7.48 (m, 2H), |
| | 7.16 (d, J = 9.6 Hz, 1H), 6.40 (q, J = 6.8 Hz, 1H), 1.89 (d, J = 7.2 Hz, 3H). HPLC purity |
| | (254 nm): 99.2%. |

### Example 3: 6-(3-Methoxyphenyl)-2-[1-(5-phenyl-4H-1,2,4-triazol-3-yl)ethyl]-2,3-dihydropyridazin-3-one (Compound 11)

A. 2-[3-(3-Methoxyphenyl)-6-oxo-1,6-dihydropyridazin-1-yl]propanenitrile. To a 50-mL round-bottom flask was placed a solution of 6-(3-methoxyphenyl)-2,3-dihydropyridazin-3-one (1 g, 4.95 mmol, as prepared in Example 1, Step A) in acetone (20 mL) then 2-chloropropanenitrile (675 mg, 7.54 mmol) and K₂CO₃ (2.07 g, 14.87 mmol) were added. The reaction was stirred at 60° C. for 16 h then the solids were filtered out and the filtrate was concentrated under reduced pressure. The residue was purified by column chromatography eluting with EtOAc/petroleum ether (1:10 up to 1:1) affording 950 mg (76%) of the title compound as an off-white solid. Mass Spectrum (LCMS, ESI pos): Calcd. for C₁₄H₁₄N₃O₂⁺: 256.1 (M+H); Found: 256.1.

B. 6-(3-Methoxyphenyl)-2-[1-(5-phenyl-4H-1,2,4-triazol-3-yl)ethyl]-2,3-dihydropyridazin-3-one. To a 2-mL vial was placed a solution of 2-[3-(3-methoxyphenyl)-6-oxo-1,6-dihydropyridazin-1-yl]propanenitrile (150 mg, 0.59 mmol, as prepared in the previous step) in tBuOH (1 mL) then benzohydrazide (40 mg, 0.29 mmol) and K₂CO₃ (4 mg, 0.03 mmol) were added. The reaction was irradiated under microwave irradiation at 150° C. for 3 h, then diluted with 30 mL of water and extracted with EtOAc (2×20 mL). The organic extracts were combined, dried over anhydrous Na₂SO₄, and concentrated under reduced pressure. The crude product was purified by Flash-Prep-HPLC (Waters:Column:X Bridge C18 OBD Prep Column, 19 mm×250 mm; Mobile Phase A: Water (0.05% TFA), Mobile Phase B: ACN; Flow rate: 20 mL/min; Gradient: 25% B to 70% B in 8 min; 254/220 nm) affording 47.5 mg (43%) of the title compound as an off-white solid. Mass Spectrum (LCMS, ESI pos): Calcd. for C₂₁H₂₀N₅O₂⁺: 374.2 (M+H); Found: 374.2. ¹H NMR (400 MHz, DMSO-d₆): δ 8.07 (d, J=10.0 Hz, 1H), 7.94 (d, J=6.8 Hz, 2H), 7.48-7.30 (m, 6H), 7.09 (d, J=10.0 Hz, 1H), 6.99-6.95 (m, 1H), 6.34 (q, J=6.8 Hz, 1H), 3.72 (s, 3H), 1.82 (d, J=7.2 Hz, 3H). HPLC purity (254 nm): 98.7%.

Using the procedure described in Example 3, with reagents, starting materials, and conditions familiar to those skilled in the art, the following compounds representative of the disclosure were prepared:

| Compound | Name and Data |
|---|---|
| 12 | 6-(3-Methoxyphenyl)-2-(1-(5-(4-(trifluoromethyl)phenyl)-4H-1,2,4-triazol-3- |
| | yl)ethyl)pyridazin-3(2H)-one. Mass Spectrum (LCMS, ESI pos): Calcd. for |
| | C₂₂H₁₉F₃N₅O₂⁺: 442.1 (M + H); Found: 442.1. ¹H NMR (300 MHz, CDCl₃): δ 8.22 (d, J = 8.4 Hz, |
| | 2H), 7.75-7.66 (m, 3H), 7.41-7.32 (m, 3H), 7.11 (d, J = 9.6 Hz, 1H), |
| | 7.00-6.97 (m, 1H), 6.39 (q, J = 7.2 Hz, 1H), 3.86 (s, 3H), 2.08 (d, J = 7.2 Hz, 3H). HPLC |
| | purity (254 nm): 98.7%. |
| 13 | 6-(3-Methoxyphenyl)-2-(1-(5-(3-(trifluoromethyl)phenyl)-4H-1,2,4-triazol-3- |
| | yl)ethyl)pyridazin-3(2H)-one. Mass Spectrum (LCMS, ESI pos): Calcd. for |
| | C₂₂H₁₉F₃N₅O₂⁺: 442.1 (M + H); Found: 442.3. ¹H NMR (300 MHz, CDCl₃): δ 8.21 (d, J = 8.1 Hz, |
| | 2H), 7.74-7.66 (m, 3H), 7.41-7.31 (m, 3H), 7.11 (d, J = 9.6 Hz, 1H), |
| | 7.00-6.97 (m, 1H), 6.40 (q, J = 7.2 Hz, 1H), 3.85 (s, 3H), 2.07 (d, J = 7.2 Hz, 3H). HPLC |
| | purity (254 nm): 99.4%. |

### Example 4: CFTR Activity Assays

i. Ussing Measurements

As discussed above, Ussing measurements are used to measure CFTR activity. In this method, primary lung epithelial cells (hBEs) homozygous for the Cystic Fibrosis-causing ΔF508 mutation are differentiated for a minimum of 4 weeks in an air-liquid interface on SnapWell filter plates prior to the Ussing measurements. Cells are apically mucus-washed for 30 minutes prior to treatment with compounds. The basolateral media is removed and replaced with media containing the compound of interest diluted to its final concentration from DMSO stocks. Treated cells are incubated at 37° C. and 5% CO₂ for 24 hours. At the end of the treatment period, the cells on filters are transferred to the Ussing chamber and equilibrated for 30 minutes. The short-circuit current is measured in voltage clamp-mode (V_{hold}=0 mV), and the entire assay is conducted at a temperature of 36° C.-36.5° C. Once the voltages are stabilized, the chambers are clamped, and data is recorded by pulse readings every 5 seconds. Following baseline current stabilization, the following additions can be applied and the changes in current and resistance of the cells can be monitored:

1. Benzamil to the apical chamber to inhibit ENaC sodium channel.

2. Forskolin to both chambers to activate ΔF508-CFTR by phosphorylation.

3. VX-770 to the apical chamber to potentiate ΔF508-CFTR channel opening.

4. CFTRinh-172 to the apical chamber to inhibit ΔF508-CFTR Cl-conductance.

The inhibitable current (that current that is blocked by CFTRinh-172) is measured as the specific activity of the ΔF508-CFTR channel, and increases in response to compound in this activity over that observed in vehicle-treated samples are identified as the correction of ΔF508-CFTR function imparted by the compound tested.

ii. hBE Equivalent Current (Ieq) Assay

Primary lung epithelial cells homozygous for the Cystic Fibrosis-causing ΔF508 mutation were differentiated for a minimum of 4 weeks in an air-liquid interface on Costar 24 well HTS filter plates prior to the equivalent current (Ieq) measurements. Cells were apically mucus-washed for 30 minutes 24 h prior to treatment with compounds. The basolateral media was removed and replaced with media containing the compound of interest diluted to its final concentration from DMSO stocks. Treated cells were incubated at 37° C. and 5% CO₂ for 24 hours. At the end of the treatment period, the media was changed to the Ieq experimental solution for 2 hours before the experiment and plates are maintained in a CO₂-free incubator during this period. The plates containing the cells were then placed in pre-warmed heating blocks at 36° C.±0.5 for 15 minutes before measurements are taken. The transepithelial voltage (V_{T}) and conductance (G_{T}) were measured using a custom 24 channel current clamp (TECC-24) with 24 well electrode manifold. The Ieq assay measurements were made following additions with standardized time periods:

1. The baseline V_{T} and G_{T} values were measured for approximately 20 minutes.

2. Benzamil was added to block ENaC for 15 minutes.

3. Forskolin plus VX-770 were added to maximally activate ΔF508-CFTR for 27 minutes.

4. Bumetanide was added to inhibit the NaK₂Cl cotransporter and shut-off secretion of chloride.

The activity data captured was the area under the curve (AUC) for the traces of the equivalent chloride current. The AUC was collected from the time of the forskolin/VX-770 addition until the inhibition by bumetanide addition. Correction in response to compound treatment was scored as the increase in the AUC for compound-treated samples over that of vehicle-treated samples.

The results are shown below in Table 1. (+ indicates activity<150% of a CFTR amplifier (Compound A) with compound at concentration shown and Compound A at 3 uM; ++ indicates activity>150% of a CFTR amplifier with compound at concentration shown and Compound A at 3 uM.

TABLE 1

| Com- | | Activity | |
|---|---|---|---|
| pound # | Structure | 3 μM | 10 μM |
| 1 | 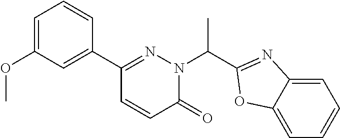 [SMILES: COc1cccc(-c2ccc(=O)n(C(C)c3nc4ccccc4o3)n2)c1] | + | + |
| 2 | 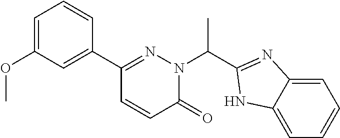 [SMILES: COc1cccc(-c2ccc(=O)n(C(C)c3nc4ccccc4[nH]3)n2)c1] | | + |
| 3 | 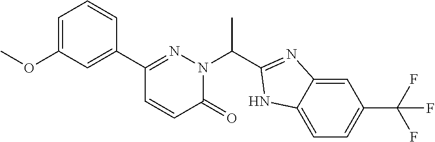 [SMILES: COc1cccc(-c2ccc(=O)n(C(C)c3nc4cc(C(F)(F)F)ccc4[nH]3)n2)c1] | ++ | ++ |
| 4 | 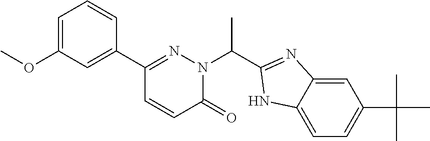 [SMILES: COc1cccc(-c2ccc(=O)n(C(C)c3nc4cc(C(C)(C)C)ccc4[nH]3)n2)c1] | + | + |
| 5 | 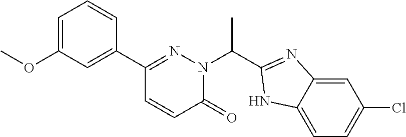 [SMILES: COc1cccc(-c2ccc(=O)n(C(C)c3nc4cc(Cl)ccc4[nH]3)n2)c1] | + | + |
| 6 | 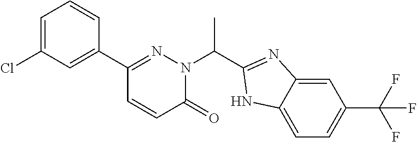 [SMILES: CC(c1nc2cc(C(F)(F)F)ccc2[nH]1)n1nc(-c2cccc(Cl)c2)ccc1=O] | ++ | ++ |
| 7 | 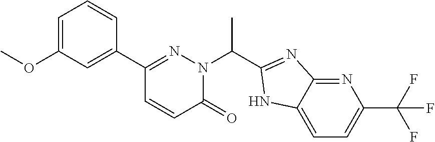 [SMILES: COc1cccc(-c2ccc(=O)n(C(C)c3nc4nc(C(F)(F)F)ccc4[nH]3)n2)c1] | + | + |
| 8 | 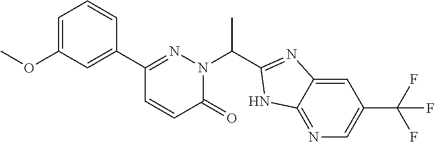 [SMILES: COc1cccc(-c2ccc(=O)n(C(C)c3nc4cc(C(F)(F)F)cnc4[nH]3)n2)c1] | + | + |
| 9 | 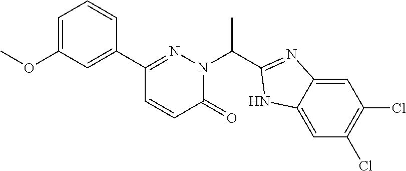 [SMILES: COc1cccc(-c2ccc(=O)n(C(C)c3nc4cc(Cl)c(Cl)cc4[nH]3)n2)c1] | ++ | ++ |
| 10 | 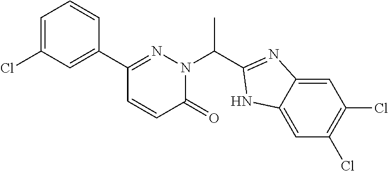 [SMILES: CC(c1nc2cc(Cl)c(Cl)cc2[nH]1)n1nc(-c2cccc(Cl)c2)ccc1=O] | + | + |
| 11 | 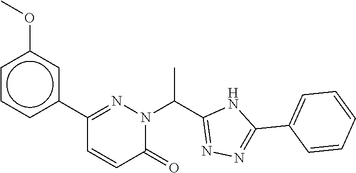 | | + |
| 12 | 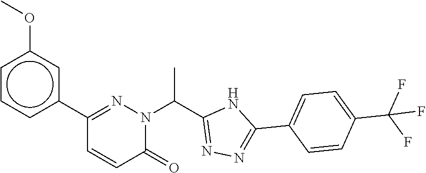 | | + |
| 13 | 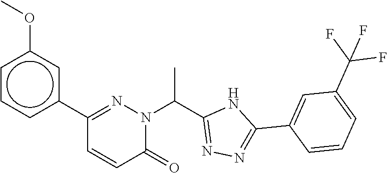 | | + |

While this disclosure has been particularly shown and described with references to embodiments thereof, it will be understood by those skilled in the art that various changes in form and details may be made therein without departing from the scope of the disclosure encompassed by the appended claims.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A compound represented by: 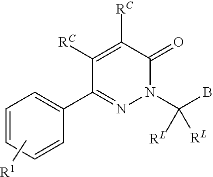
or pharmaceutically acceptable salts and/or stereoisomers thereof, wherein:
Ring B is selected from the group consisting of a 5-6 membered monocyclic heteroaryl having 1, 2 or 3 heteroatoms each independently selected from the group consisting of S, N, NR^{a} and O; a 9-10 membered bicyclic heteroaryl having 1, 2 or 3 heteroatoms each selected from the group consisting of S, N, NR^{a} and 0;
R¹ independently for each occurrence, is selected from the group consisting of hydrogen, halogen, hydroxyl, cyano, C₁₋₆alkyl, C₂₋₆alkenyl, C₂₋₆alkynyl, C₃₋₆cycloalkyl, heterocycle, C₁₋₆alkoxy, —NR^{a}R^{b}, phenyl, benzyl, and —O-phenyl;
Ring B is optionally substituted by one, two, three or four substituents each selected from R⁶;
R⁶ is, independently for each occurrence, selected from the group consisting of halogen, hydroxyl, cyano, C₁₋₆alkyl, C₂₋₆alkenyl, C₂₋₆alkynyl, C₃₋₆cycloalkyl, heterocycle, R⁶⁶, C₁₋₆alkoxy, and —NR^{a}R^{b};
R⁶⁶ is phenyl or a 5 membered heteroaryl with 1, 2 or 3 heteroatoms each selected from O, S, and N, wherein R⁶⁶ is optionally substituted with one or two substituents each selected from halogen, hydroxyl, cyano, C₁₋₆alkyl, C₂₋₆alkenyl, C₂₋₆alkynyl, C₁₋₆alkoxy, C₃₋₆cycloalkyl, and heterocycle;
R^{C} is independently selected for each occurrence from the group consisting of H, halogen, hydroxyl, C₁₋₆alkyl, C₁₋₆alkoxy, C₃₋₆cycloalkyl, phenyl and —O-phenyl;
R^{L} is independently selected for each occurrence from the group consisting of H, methyl, ethyl, propyl, butyl, cyclopropyl, cyclobutyl, cyclohexyl, cyclopentyl, heteroaryl, heterocycle, phenyl and benzyl;
R^{a} is independently selected for each occurrence from the group consisting of H, C₁₋₃alkyl, C₃₋₆ cycloalkyl, phenyl, and C(O)—C₁₋₃alkyl;
R^{b} is independently selected for each occurrence from the group consisting of H and C₁₋₃alkyl;
or R^{a} and R^{b} taken together with the nitrogen to which they are attached form a 3-6 membered heterocyclic ring; and
wherein C₁₋₆alkyl, C₂₋₆alkenyl, C₂₋₆alkynyl, C₃₋₆cycloalkyl, C₁₋₆alkoxy, C₃₋₆cycloalkyl, heteroaryl, heterocycle, benzyl and phenyl are each optionally substituted by one, two or three substituents each independently selected from halogen, cyano, methyl, methoxy, carboxy, C(O)—O—C₁₋₃ alkyl, C(O)—C₁₋₃alkyl phenyl, —NR^{a}R^{b}, S(O)_{w}-methyl (where w is 0, 1 or 2), —S(O)_{w}—NR^{a}R^{b}(where w is 0, 1 or 2), and —NR^{b}—S(O)_{w} (where w is 0, 1, or 2), and hydroxyl.

2. The compound of claim 1, wherein ring B is: 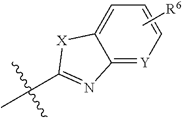
wherein X is O or NR^{a} and Y is CH or N.

3. The compound of claim 1 or 2, wherein ring B has at least one N member and one NR^{a} member.

4. The compound of claim 1, wherein ring B is:

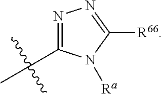

5. The compound of claim 4, wherein R⁶⁶ is phenyl or 5 membered heteroaryl.

6. The compound of any one of claims 1-5, wherein one R^{L} is H and one R^{L} is methyl.

7. The compound of any one of claims 1-6, wherein R^{C} for each occurrence is selected from H and halogen.

8. The compound of any one of claims 1-7, wherein B is selected from the group consisting of:

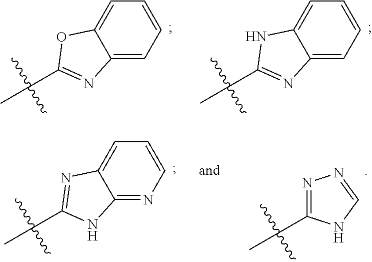 [SMILES: *c1nc2ccccc2o1 ; *c1nc2ccccc2[nH]1 ; *c1nc2cccnc2[nH]1 ; *c1nnc[nH]1]

9. The compound of claim 1, selected from the group consisting of: 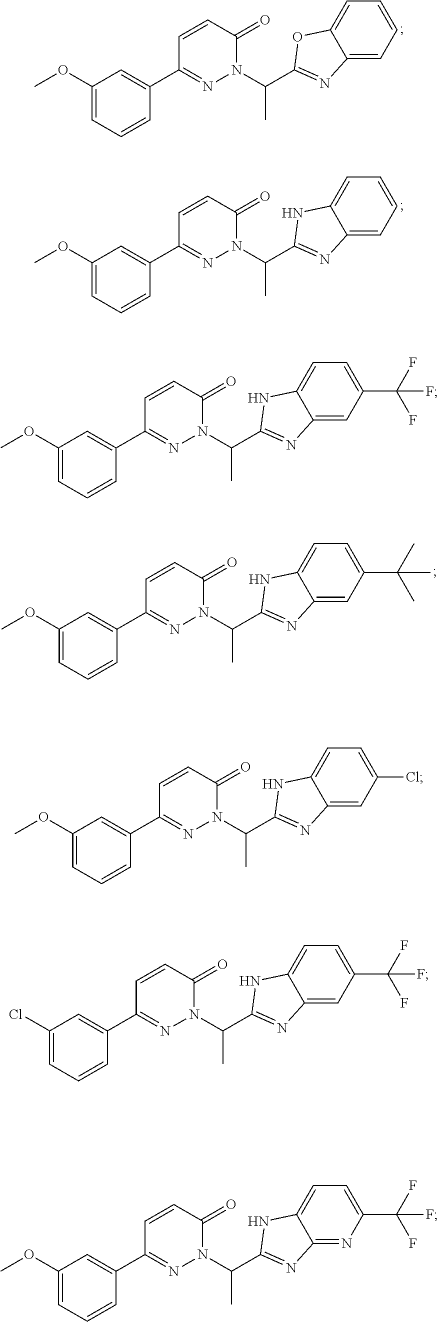 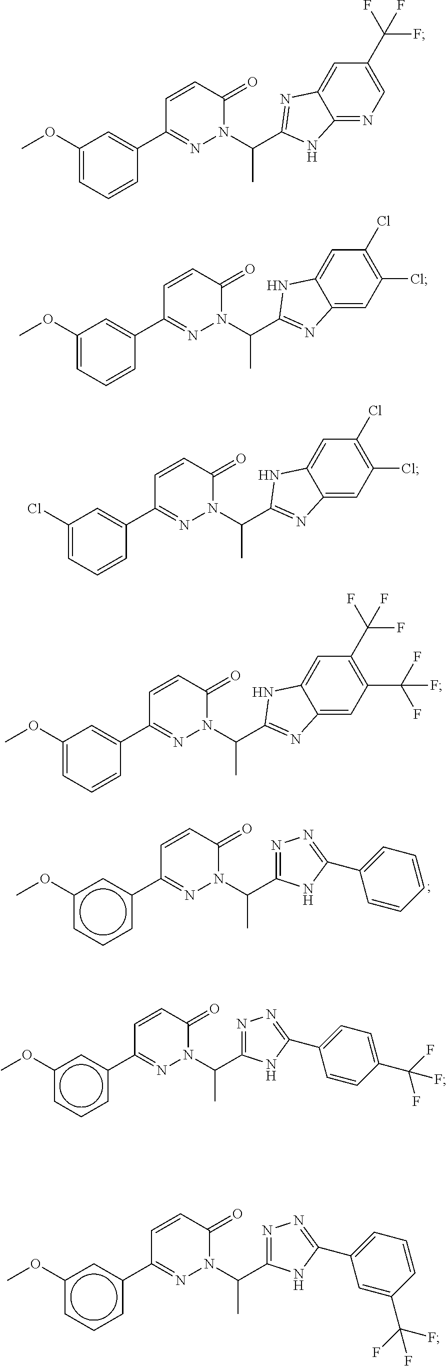 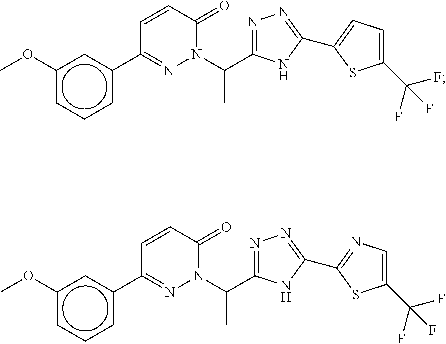
pharmaceutically acceptable salt or stereoisomer thereof.

10. A pharmaceutically acceptable composition comprising a compound of any one of claims 1-9, and a pharmaceutically acceptable excipient.

11. A method for modulating or enhancing a cystic fibrosis transmembrane conductance regulator in a patient in need thereof, comprising administering to the patient an effective amount of a composition of claim 10 or a compound of any one of claims 1-9.

12. The method of claim 11, wherein the cellular processing of a mutant CFTR is enhanced.

13. The method of claim 12, wherein the mutant CFTR is selected from the group consisting ΔF508, S549N, G542X, G551D, R117H, N1303K, W1282X, R553X, 621+1G>T, 1717-1G>A, 3849+10kbC>T, 2789+5G>A, 3120+1G>A, I507del, R1162X, 1898+1G>A, 3659delC, G85E, D1152H, R560T, R347P, 2184insA, A455E, R334W, Q493X, and 2184delA CFTR.

14. The method of claim 13, wherein ΔF508 CFTR activity is enhanced.

15. The method of any one of claims 11-14, wherein the patient is suffering from a disease associated with decreased CFTR activity.

16. The method of claim 15, wherein the disease is selected from the group consisting of cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, A-β-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, Sjogren's syndrome, familial hypercholesterolemia, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, nephrogenic DI, Charcot-Marie Tooth syndrome, Perlizaeus-Merzbacher disease, Alzheimer's disease, Parkinson's disease, amyotrophic lateral sclerosis, progressive supranuclear palsy, Pick's disease, Huntington's disease, spinocerebellar ataxia type I, spinal and bulbar muscular atrophy, dentatorubral pallidoluysian, myotonic dystrophy, hereditary Creutzfeldt-Jakob disease (due to prion protein processing defect), Fabry disease, cholestatic liver disease (e.g. Primary biliary cirrhosis (PBC) and primary sclerosing cholangitis (PSC)), and Straussler-Scheinker syndrome.

17. The method of claim 16, wherein the disease is cystic fibrosis.

18. The method of any one of claims 11-17, wherein the patient is human.

19. The method of any one of claims 11-18, further comprising administering at least one or two additional CFTR modulators.
